(19) United States
(12) Patent Application Publication
(10) Pub. No.: US 2026/0219254 A1
(43) Pub. Date: Jul. 30, 2026

(54) WATER QUALITY PLATFORM AND COSMETIC ECOSYSTEM

(71) Applicant: L'Oreal, Paris (FR)

(72) Inventors: Nicholas Sinclair, San Diego, CA (US); Fred Orsita, Wayne, NJ (US); Dominic Akerele, Brooklyn, NY (US); Odessa Xinyao Deng, Hoboken, NJ (US)

(73) Assignee: L'Oreal, Paris (FR)

(21) Appl. No.: 19/041,700

(22) Filed: Jan. 30, 2025

(51) Int. Cl. G01N 33/18; G01N 31/22

(52) U.S. Cl. CPC G01N 33/1813; G01N 31/221; G01N 33/1853

(57) ABSTRACT

Systems for leveraging water quality data for the determination and recommendation of haircare products, services, and lifestyle decisions. A system configured for management of hair health of an individual includes circuitry configured to receive a measurement of one or more water quality markers that are relevant to the individual and compute, based on the measurement, whether a product or a service would advance the hair health of the individual toward a desired beauty outcome for a responsiveness metric; and circuitry configured to recommend or not recommend the product or the service to the individual, based on the responsiveness metric, such that the hair health of the individual is advanced toward the desired beauty outcome.

### SUMMARY

In an aspect, the disclosure provides a system configured for management of hair health of an individual, the system comprising: circuitry configured to receive a measurement of one or more water quality markers that are relevant to the individual and compute, based on the measurement, whether a product or a service would advance the hair health of the individual toward a desired beauty outcome for a responsiveness metric; and circuitry configured to recommend or not recommend the product or the service to the individual, based on the responsiveness metric, such that the hair health of the individual is advanced toward the desired beauty outcome.

In embodiments, the measurement of the one or more water quality markers is transmitted from a data storage computational device and received by a computational device.

In embodiments, the data storage computational device comprises associations of a plurality of geophysical locations and corresponding measurements of water quality markers at or near the geophysical locations, or at or near a water source thereof.

In embodiments, the computational device comprises a networked server, a cloud computing system, a smartphone, a tablet, a desktop computer, a laptop computer, a smart watch, a wearable device, or any combination thereof.

In embodiments, a first threshold of the measurement corresponds with a positive responsiveness metric and predicted responsiveness of hair of the individual to the product or the service, and wherein a second threshold of the measurement corresponds with a negative responsiveness metric and predicted non-responsiveness of hair of the individual to the product or the service.

In embodiments, the water quality marker comprises water hardness, and the measurement comprises a concentration of magnesium (Mg²⁺) and/or a concentration of calcium (Ca²⁺) in water that is relevant to the individual.

In embodiments, the product comprises a cosmetic mitigation product configured to mitigate hair damage due to water hardness, and wherein the cosmetic mitigation product comprises a chelating agent configured to chelate magnesium and/or calcium for removal from the water.

In embodiments, the cosmetic mitigation product is a shampoo, conditioner, deep conditioner, hair mask configured for moisture replenishment and/or damage repair, and/or scrub primer; and wherein the chelating agent is selected from the group including, but not necessarily limited to: disodium ethylenediaminetetraacetic acid (EDTA), tetrasodium EDTA, pentasodium pentetate, sodium gluconate, sodium phytate/phytic acid, citric acid, glycine, and any combination thereof.

In embodiments, the product comprises a lifestyle mitigation product configured to mitigate hair damage due to water hardness, and wherein the lifestyle mitigation product comprises a chelating agent configured to chelate magnesium and/or calcium for removal from the water.

In embodiments, the lifestyle mitigation product is a water softener, a shower stick, or a recommendation to reduce a frequency of hair washing and/or hair rinsing; and wherein the chelating agent is selected from the group including, but not necessarily limited to: disodium EDTA, tetrasodium EDTA, pentasodium pentetate, sodium gluconate, sodium phytate/phytic acid, citric acid, glycine, and any combination thereof.

In embodiments, the one or more water quality metrics are relevant to the individual due at least in part to the measurement being taken from water that is or will be used by the individual at a particular time.

In embodiments, the water is or will be proximal to the individual at the particular time in the course of travel or transportation.

In embodiments, the water quality marker comprises pH, chlorine (Cl⁻), copper (I) (Cu⁺), copper (II) (Cu²⁺), or any combination thereof, and the measurement comprises a pH level, a concentration of chlorine (Cl⁻), a concentration of copper (I) (Cu⁺), a concentration of copper (II) (Cu²⁺), or any combination thereof, in water that is relevant to the individual.

In embodiments, the system is configured to receive the measurement from a water testing kit that is configured for testing one or more water quality markers that are relevant to the individual.

In embodiments, the water testing kit comprises one or more pH colorimetric strips for measurement of pH, and/or one or more reagents for measurement of at least one water quality marker selected from the group including, but not necessarily limited to: magnesium (Mg²⁺), calcium (Ca²⁺), chlorine (Cl⁻), copper (I) (Cu⁺), copper (II) (Cu²⁺), and any combination thereof.

This summary is provided to introduce a selection of concepts in a simplified form that are further described below in the Detailed Description. This summary is not intended to identify key features of the claimed subject matter, nor is it intended to be used as an aid in determining the scope of the claimed subject matter.

### DESCRIPTION OF THE DRAWINGS

FIG. 1 shows an overview of example water quality markers that are relevant to individuals' skin and hair health, as well as their impact thereon, according to aspects of the disclosure.

FIG. 2 shows a flow chart for an example method for management of hair health of an individual, which can be implemented by one or more computational devices and/or systems, according to aspects of the disclosure.

FIG. 3A shows a user interface of an example system configured to receive water quality marker measurements and make recommendations to a user, as well as display water quality and hair and skin health impacts that are based on geolocation, according to aspects of the disclosure.

FIG. 3B shows a user interface of an example system configured to receive water quality marker measurements and make recommendations to a user, as well as display water quality and hair and skin health impacts that are based on geolocation, according to aspects of the disclosure.

FIG. 3C shows a user interface of an example system configured to receive water quality marker measurements and make recommendations to a user, as well as display a banner ad for the user to obtain additional information about water quality and make informed choices for hair and skincare in view of the water quality or hardness, according to aspects of the disclosure.

FIG. 4 shows a schematic diagram of an example computational device that can be all or part of a system configured to receive water quality marker measurements and make recommendations to a user, according to aspects of the disclosure.

The foregoing aspects and many of the attendant advantages of this disclosure will become more readily appreciated as the same become better understood by reference to the following detailed description, when taken in conjunction with the accompanying drawings and annotations as listed at Table 1.

### DETAILED DESCRIPTION

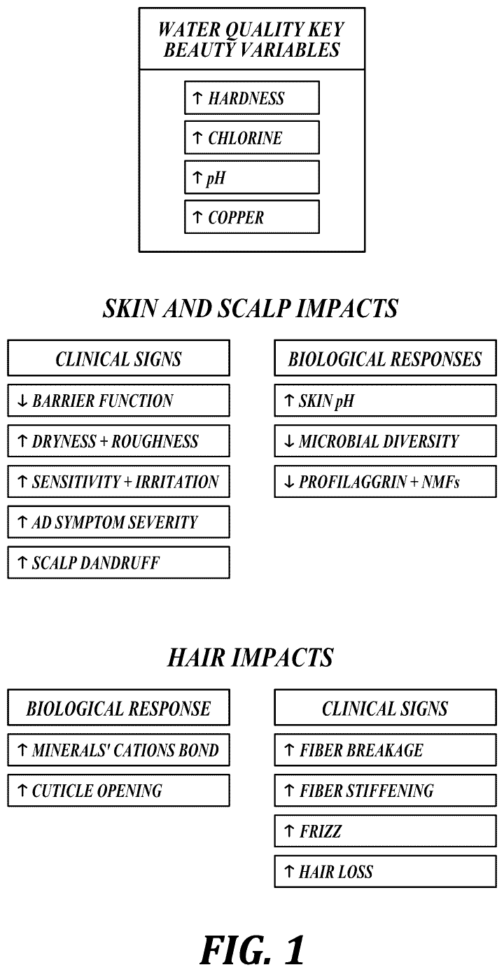

Higher levels of water hardness result in buildup of minerals in the hair, which is generally detrimental to hair and scalp health and can negatively impact the ability of the skin and scalp to function as a barrier. Mineral buildup can also decrease microbial diversity, decrease profilaggrin, and natural moisturizing factors (NMFs), and can also increase dryness, roughness, sensitivity, irritation, atopic dermatitis (AD) symptom severity, scalp dandruff, skin pH, minerals' cations bond, cuticle opening, fiber breakage, fiber stiffening, frizz, and hair loss. Water quality markers that contribute to water hardness include chlorine levels, pH, and copper levels (e.g., copper (I), copper (II), or both) (FIG. 1 and Table 2).

Prior efforts to manage water hardness have generally involved relatively permanent installations of water softener systems, or other water treatment systems, at the point of entry of a source of water into a building, such as a home or office, or at a water tank of the building. These systems can malfunction for a period, during which time the water hardness inside the building may increase. During the time before the system is repaired or replaced, individuals using water inside the building to bathe would be at increased risk of detrimental hair and skin health due to high water hardness. In addition, even when these systems are operable, individuals may travel to locations without any water softener system installed, such as a gym, a hotel or rental unit, or the like. During this travel away from home, elevated water hardness in the water at these locations can be detrimental to hair and skin health.

As such, there is a need for approaches that leverage water quality data for recommendation of high-quality and effective products and services for management of water hardness, including at the individual level, that can be achieved independent of location or travel status. The present disclosure addresses these and other long-felt and unmet needs in the art.

Accordingly, in an aspect, the disclosure provides a system configured for management of hair health of an individual. The system comprises circuitry configured to receive a measurement of one or more water quality markers that are relevant to the individual and compute, based on the measurement, whether a product or a service would advance the hair health of the individual toward a desired beauty outcome for a responsiveness metric. The responsiveness metric can be predictive of a positive response to the product or service such that the product or service would be beneficial or effective, or can be predictive of a negative response to the product or service such that the product or service may not be as beneficial or effective as desired. The responsiveness metric can be computationally compared with one or more thresholds for determination of predicted responsiveness as the result of surpassing a first threshold for a positive responsiveness metric (predicted responsiveness of hair to the product or the service) and a second threshold for a negative responsiveness metric (predicted non-responsiveness of hair to the product or the service). In that regard, the system can comprise circuitry configured to recommend or not recommend the product or the service to the individual, based on the responsiveness metric, such that the hair health of the individual is advanced toward the desired beauty outcome regardless of whether a particular product or service is recommended or selected.

In embodiments, a system utilizes highly accurate tap water data sets, optionally in combination with data from one or more water test kits, to enable new services for consumers and business customers, such as salons. Systems can utilize data obtained from a tap water data platform for the enablement of Internet or online experiences in which a consumer or beauty business customer shares an approximate location (e.g., postal code) or exact geolocation into a digital form (e.g., “Where do you live?”), which the system uses for computation of various output. Examples of such outputs include, but are not limited to: a quantitative assessment of localized tap water conditions that can be displayed visually by a computational device for viewing and interpretation; one or more water quality scores representing the effects of local tap water conditions on consumers' scalp, skin, and hair beauty or health; personalized recommendations including cosmetic products, water products (e.g., filtration units or in-line water softener devices), and lifestyle habits based on a consumer profile and/or individuals' responses to questions about their beauty habits and concerns, lifestyle, and/or demographics, and/or results of tap water assessments, and the like.

The disclosure also provides a tap water testing kit and/or tap water testing service in which a suite of tests, such as pH level as can be determined by pH colorimetric strips or water samples that can be mailed out for laboratory testing, can be used for the generation of real-time or de novo water quality data. Tap water testing kits and services can also enable consumers and beauty business customers to receive the most accurate and detailed information about their tap water and its effects on beauty; for example, in embodiments, the tap water data and results from a testing kit service can be combined, such that an individual can use a system of the disclosure to at least approximately determine their local tap water quality via geolocation, and can be offered the option to test their tap water directly with a testing kit to learn additional information about water quality and how it impacts hair and skin health and beauty.

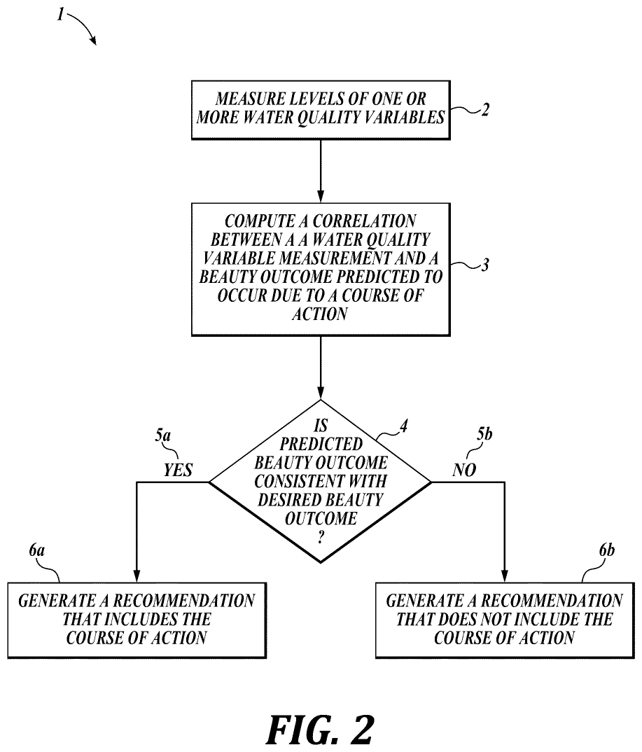

An example method 1 for management of hair health of an individual is shown at FIG. 2. As a first step, levels of one or more water quality variables are measured 2, either by an individual using a system of the disclosure or a third party, such as a company or governmental entity responsible for monitoring water quality and/or collecting and distributing water quality data, for example. At step 3, a correlation between a water quality variable measurement and a beauty outcome that is predicted to occur due to a course of action is computed. At step 4, a determination is made as to whether the predicted beauty outcome is consistent with a desired beauty outcome; if “YES” (step 5a: YES), then the method proceeds to step 6a at which a recommendation that includes the course of action is generated and/or made to the individual; if “NO” (step 5b: NO), then the method proceeds to step 6b at which a recommendation that does not include the course of action is generated and/or made to the individual.

In embodiments, a recommendation that does not include a particular course of action can include an alternative course of action, which can be the result of an alternative or additional iteration of the method. For example, if the water quality variable is measured and the system computes that water hardness may impact hair or skin health, the system may suggest the use of a shampoo that comprises one or more chelating agents for removal of metals or metal-containing compounds from the water during use shampooing the hair and scalp. However, if the individual is not interested in the shampoo (e.g., as computed by the system as a result of data regarding or input from the individual), then an alternative product can be suggested, such as a shower stick or in-line water softening device. In this manner, the system does not merely suggest products or services, but provides valuable options to the individual to make informed decisions for hair and skin health that are data driven.

In embodiments, variables other than measured water quality can be taken into consideration by the system as part of the computation of a recommendation. For example, the system can utilize data that pertains to other environmental factors, such as ultraviolet (UV) light exposure or weather data, the individual or their particular hair or skin or situation, such as natural and/or artificial hair color or status, current haircare and/or skincare routines such as hair washing frequency, current products and/or additives used, baseline water quality (e.g., water quality that is averaged or that pertains to a regular or long-term location or water source of the individual, such as their home), beauty concerns, desired beauty outcomes or results, desired price range, and/or ingredient preferences (e.g., sulfate-free, organic, or the like), for example.

In embodiments, a system of the disclosure can compute hair and/or skin health hazard or risk of damage based on a plurality of variables, and can identify (at least potentially) synergistic interactions between multiple variables as part of the computation. For example, if copper (I) and/or copper (II) levels are higher than a threshold value, in combination with estimated or actual UV light exposure being above a threshold value, then the system can compute a risk of a synergistic effect of copper and UV light exposure in contributing to poor hair and/or skin health. In this manner, a score or metric can be computed by the system and used by the system for the computation of recommendations.

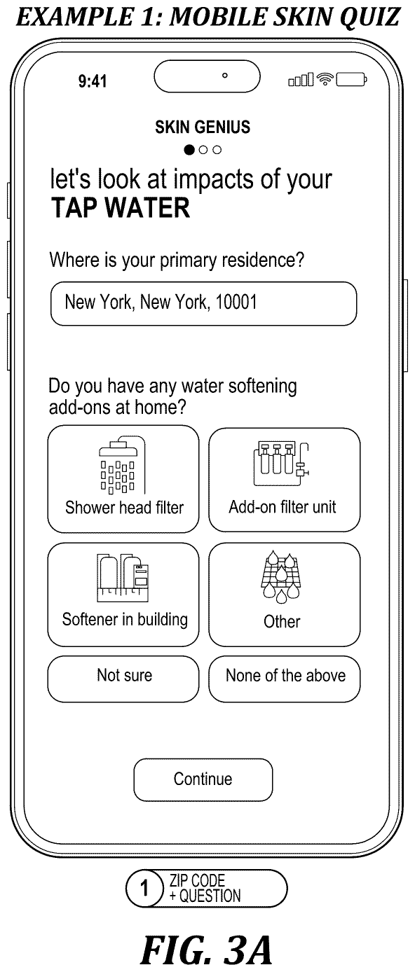

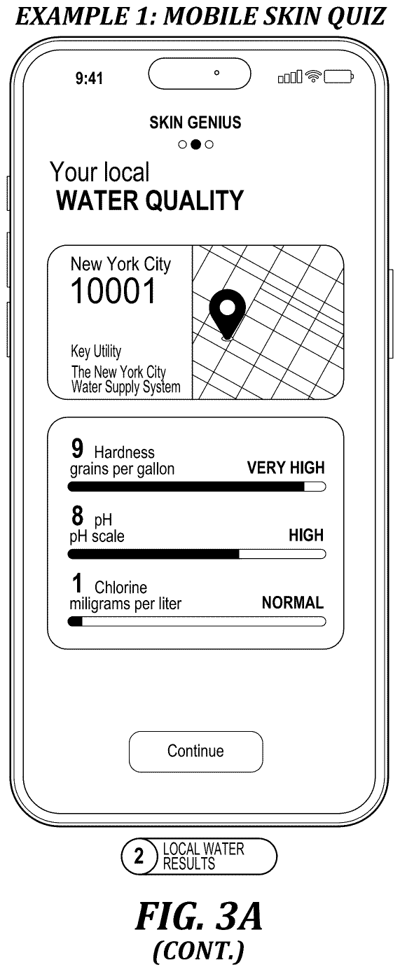

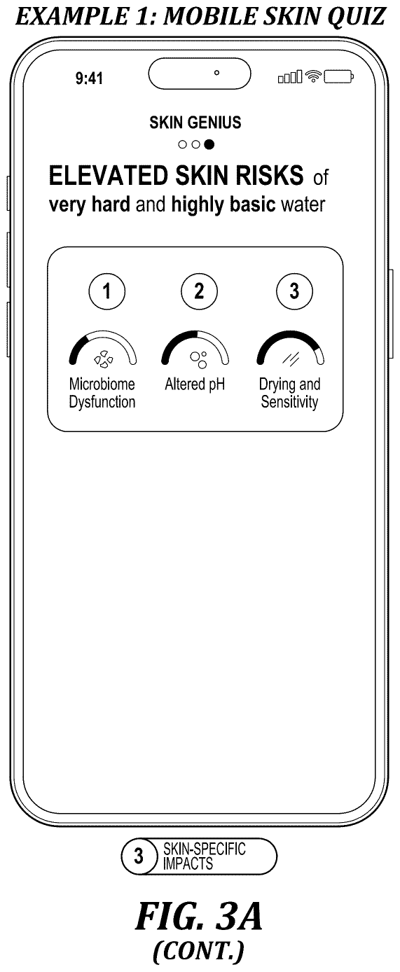

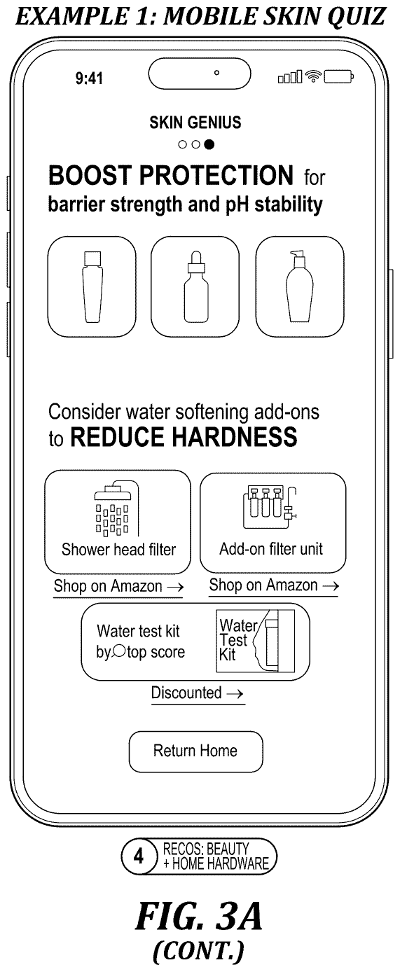

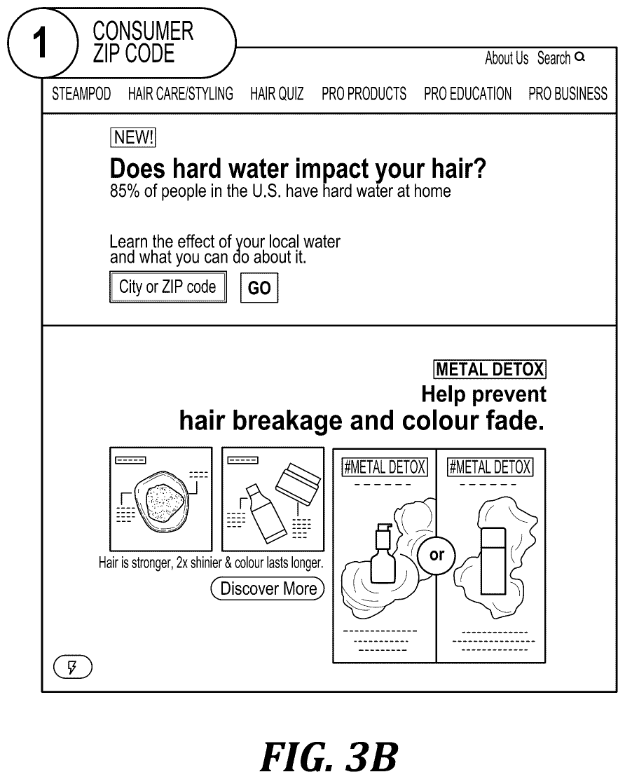

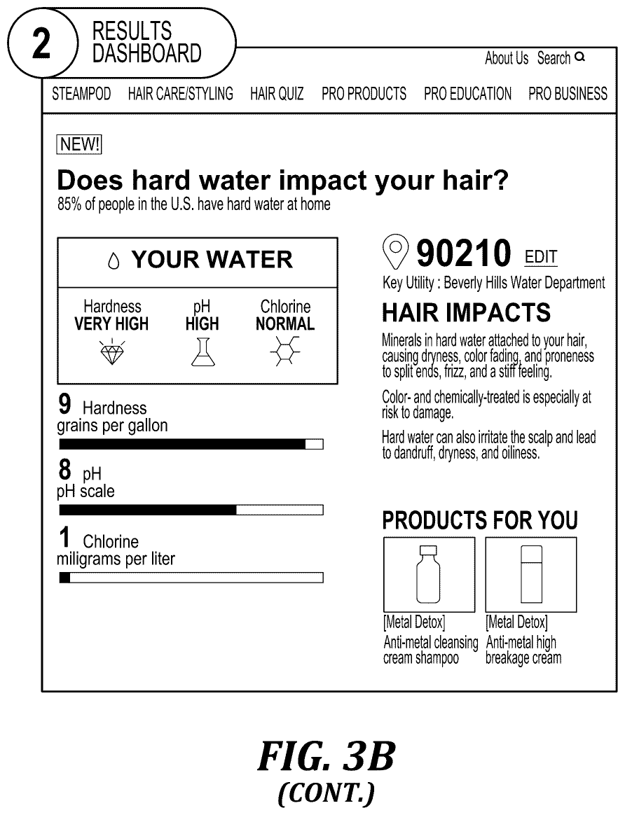

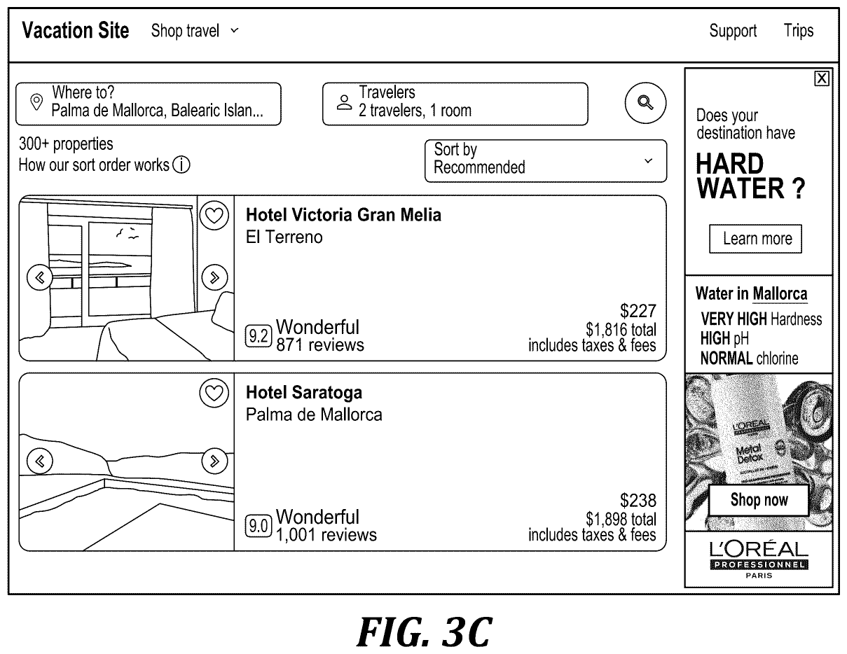

In embodiments, a recommendation of a product or service can be made as a suggestion, or as an option, presented to the individual by way of a computational device of a system, as can occur during an Internet-based web session of the computational device with a travel planning website, a hotel website, an airline website, a train or bus service website, a car rental website, or the like. In example embodiments, the purchase of a product or service can be made by an individual during their booking tickets for travel, as can occur with Internet-based commerce; with this approach, the product or service can be added to the purchase order and made available to the individual at the time of travel (see, e.g., FIGS. 3A-3C). For example, with the addition, hotel staff can add a cosmetic mitigation product as a metal chelating shampoo with purchase of a hotel stay, and the shampoo be placed in a hotel room in advance of the individual arriving for the option of use upon their arrival, if the system computes the water hardness level as being higher than desired.

The disclosure provides methods, and systems configured for the performance of methods. Systems can comprise any number or type of computational devices that are arranged in any suitable configuration for operation. In embodiments, a measurement of the one or more water quality markers is transmitted from a data storage computational device and received by a computational device. The data storage computational device can be comprised of one or more networked servers having stored thereon data that pertains to water quality across a spectrum of geophysical locations, for example. In embodiments, the data storage computational device comprises associations of a plurality of geophysical locations and corresponding measurements of water quality markers at or near the geophysical locations, or at or near a water source thereof, such as a public water source. The computational device that receives the measurement can be comprised of a personal device such as a smartphone operably connected to the data storage computational device by way of a network connection, for example. In embodiments, the computational device comprises a networked server, a cloud computing system, a smartphone, a tablet, a desktop computer, a laptop computer, a smart watch, a wearable device, or any combination thereof.

In addition, while any water quality metric can be used by a system and method of the disclosure, in example embodiments, the water quality marker comprises water hardness, and the measurement comprises a concentration of magnesium (Mg²⁺) and/or a concentration of calcium (Ca²⁺) in water that is relevant to the individual. In embodiments, the measurement comprises a concentration of magnesium and a concentration of calcium in the water, as a combined indicator of water hardness. In a non-limiting example, concentration can be expressed in milligrams per liter (mg/L) or grains per gallon (gpg), e.g., as calcium carbonate, while water hardness can be calculated from the equation Hardness_{WATER}=X[Ca²⁺]+Y[Mg²⁺], wherein X and Y are weight factors (e.g., X=2.497 and Y=4.118, in which case fluctuations in magnesium concentration can affect water hardness more significantly than fluctuations in calcium concentration).

In embodiments, the product comprises a cosmetic mitigation product configured to mitigate hair damage due to water hardness, and the cosmetic mitigation product comprises a chelating agent configured to chelate magnesium and/or calcium for removal from the water. Any suitable chelating agent can be implemented, however, non-limiting examples of chelating agents that can be incorporated into the product include disodium ethylenediaminetetraacetic acid (EDTA), tetrasodium EDTA, pentasodium pentetate, sodium gluconate, sodium phytate/phytic acid, citric acid, glycine, and any combination thereof. In embodiments, the cosmetic mitigation product is a shampoo, conditioner, deep conditioner, hair mask configured for moisture replenishment and/or damage repair, and/or scrub primer. In embodiments, the product comprises a lifestyle mitigation product configured to mitigate hair damage due to water hardness, and the lifestyle mitigation product comprises a chelating agent configured to chelate magnesium and/or calcium for removal from the water. Any suitable chelating agent can be implemented, however, non-limiting examples of chelating agents that can be incorporated into the product include disodium EDTA, tetrasodium EDTA, pentasodium pentetate, sodium gluconate, sodium phytate/phytic acid, citric acid, glycine, and any combination thereof. In embodiments, the lifestyle mitigation product is a water softener, a shower stick, or a recommendation to reduce a frequency of hair washing and/or hair rinsing (e.g., a prompt or message provided by the system), for example. In embodiments, a shower stick comprises a portable water softener device that is attachable to a water nozzle of a shower, bathtub, or sink, for in-stream water softening prior to use of the water.

In embodiments, the one or more water quality metrics are relevant to the individual due at least in part to the measurement being taken from water that is or will be used by the individual at a particular time. For example, if the individual is using a computational device to plan travel to a location, the computational device can be configured to display water quality metrics that are relevant to the location at the time of travel (FIGS. 3A-3C). As a result of this display of water quality metrics, the individual can query the system for additional information about options for managing water quality during travel. The response of the system can comprise one or more product recommendations, one or more service recommendations, and/or one or more lifestyle recommendations. In embodiments, the water is or will be proximal to the individual at A particular time in the course of travel or transportation, for example, during a hotel stay or a visit to another city or country.

While any suitable water quality marker can be used, in embodiments, the water quality marker relates to water hardness and can comprise pH, chlorine (Cl⁻), copper (I) (Cu⁺), copper (II) (Cu²⁺), or any combination thereof, and the measurement comprises a pH level, a concentration of chlorine (Cl⁻), a concentration of copper (I) (Cu⁺), a concentration of copper (II) (Cu²⁺), or any combination thereof, in water that is relevant to the individual. The measurement can be made in advance of a use of a system of the disclosure, for example, by a third party who collects or analyzes water quality data from one or more additional sources. In addition, or as an alternative, the measurement can be made by an individual using a kit of the disclosure to test water at a location that is relevant to them. As such, in embodiments, the system is configured to receive the measurement from a water testing kit that is configured for testing one or more water quality markers as described herein.

In embodiments, a water testing kit comprises one or more pH colorimetric strips for measurement of pH, and/or one or more reagents for measurement of at least one water quality marker selected from the group including, but not necessarily limited to: magnesium (Mg²⁺), calcium (Ca²⁺), chlorine (Cl⁻), copper (I) (Cu⁺), copper (II) (Cu²⁺), and any combination thereof. A water testing kit can comprise an instructional material in addition to one or more reagents for water testing, such that a user is enabled in the correct use of the kit to sample and test water quality.

### Tables

TABLE 1
Drawings elements and descriptions.

| Reference | Description |
|---|---|
| 1 | Method for management of hair health of an individual |
| 2 | Measure levels of one or more water quality variables |
| 3 | Compute a correlation between a water quality |
| | variable measurement and a beauty outcome |
| | predicted to occur due to a course of action |
| 4 | Is predicted beauty outcome consistent with |
| | desired outcome? |
| 5a | Predicted beauty outcome consistent with |
| | desired outcome: YES |
| 5b | Predicted beauty outcome consistent with |
| | desired outcome: NO |
| 6a | Generate a recommendation that includes the |
| | course of action |
| 6b | Generate a recommendation that does not |
| | include the course of action |
| 7 | Computational device |
| 8 | System memory |
| 9 | Processor |
| 10 | Storage medium |
| 11 | Network interface |
| 12 | Communication bus |

TABLE 2
Summary of water hardness information relevant to the disclosure.

| | |
|---|---|
| Scale | Hardness (magnesium, calcium) |
| (measurements/ | Soft: 0-60 milligrams per liter (mg/L) as calcium |
| categorizations) | carbonate |
| | Moderately hard: 61-120 mg/L as calcium |
| | carbonate |
| | Hard: 121-180 mg/L as calcium carbonate |
| | Very hard: more than 180 mg/L as calcium |
| | carbonate |
| Hair Impact | Hard water reduces tensile strength of hair, which |
| Description | makes hair more brittle and prone to breakage; |
| (mechanisms of | Perceived poor hair health = higher metal levels in |
| action and | hair; Hard water causes reduced foamability, |
| impacts on hair | rinsability, and cleansing efficiency; Fades color- |
| quality) | treated hair; makes dull |
| Hair Impact | HIGH IMPACT |
| Rating | Virgin Hair: Medium impact |
| (overall level of | Slightly Damaged Hair: High impact |
| impact on hair | Very Damaged Hair: Higher impact |
| quality + relative | |
| impact based on | |
| other factors) | |
| Hair Concerns | Brittleness, breakage, frizz, dry, dull, stiff |
| (succinct list of | (decreased elasticity), increased friction and |
| symptoms) | porosity, loss and thinning, color change |
| | (accelerated fading, brassiness) |
| Cosmetic | Chelating/Clarifying Hair Care (contains |
| Mitigation | chelants: Disodium EDTA, Tetrasodium EDTA, |
| (product | Pentasodium Penetrate, Sodium Gluconate, |
| recommendations | Sodium Phytate/Phytic Acid, CITRIC ACID) - |
| to treat/correct | Shampoo, Conditioner, Mask, Scrub Primer; Deep |
| tensions) | Conditioner/Hair Masks (to replenish moisture |
| | and repair damage); Dual citric acid + glycine |
| | (attact Ca and then fill in microbreaks) |
| Lifestyle | Water Softener, Showersticks; Reduce frequency |
| Mitigation | of wash/rinse time |
| (lifestyle choices | |
| to treat/correct | |
| tensions) | |

### Terminology

As used herein, including in the claims, the terms “a,” “an,” “the,” and the like, refer to the singular and the plural forms of the object or element referenced. The term “comprising” is inclusive or open-ended and does not exclude additional, unrecited elements or steps. The term “consisting of,” as used in a claim, excludes any element, step, or ingredient not specified in the claim. The term “consisting essentially of,” as used in a claim, limits the scope of the claim to the specified materials or steps and those that do not materially affect the basic and novel characteristic(s) of the claim.

The present application may include references to directions, such as “vertical,” “horizontal,” “front,” “rear,” “left,” “right,” “top,” and “bottom,” etc. These references, and other similar references in the present application, are intended to assist in helping describe and understand the particular embodiment (such as when the embodiment is positioned for use) and are not intended to limit the present disclosure to these directions or locations.

The present application may also reference quantities and numbers. Unless specifically stated, such quantities and numbers are not to be considered restrictive, but exemplary of the possible quantities or numbers associated with the present application. Also in this regard, the present application may use the term “plurality” to reference a quantity or number. In this regard, the term “plurality” is meant to be any number that is more than one, for example, two, three, four, five, etc. The term “about,” “approximately,” etc., means plus or minus 5% of the stated value. The term “based upon” means “based at least partially upon.”

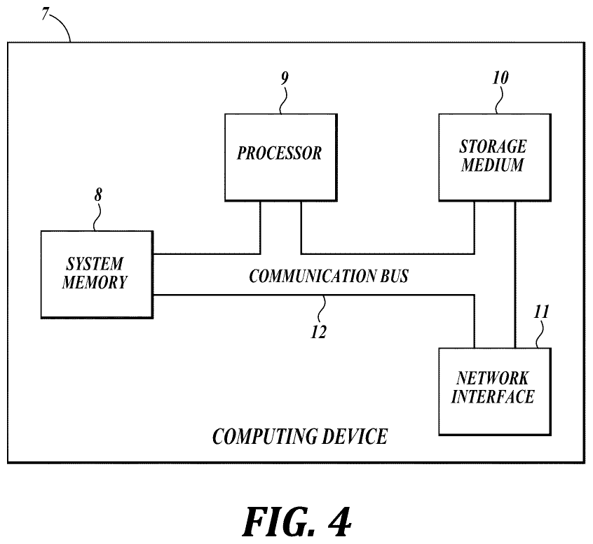

As used herein, the term “computational device,” and similar terms, refers to an electronic computational device that is configured, or that can be configured, to perform all or part of a method or process of the disclosure (see, e.g., computational device 7 of FIG. 4). The term refers to a physical hardware computing device that is configured for performing all or part of any method of the disclosure, in any order or sequence of steps, optionally with human input. In embodiments, circuitry (e.g., system memory 8, processor 9, storage medium 10, network interface 11, communication bus 12) of such a computational device is configurable with a processor 9 and processor-executable instructions stored on a non-transitory machine-readable medium 10 of the computational device 7, as a non-limiting example, but other approaches for configuring circuitry of the computational device can be implemented in embodiments. In embodiments, the computational device includes a processor 9 for execution of instructions stored on a non-transitory machine-readable medium 10, for enabling the processor 9 to carry out all or part of a method or process of the disclosure. Accordingly, in embodiments, a computational device 7 includes a software application configured to perform all or part of one or more methods or processes of the disclosure, in any order or combination. However, in embodiments, a computational device 7 includes dedicated hardware circuitry. Further configuration of circuitry of the computational device can include networking circuitry 11, for example, circuitry configured for a wireless connection, such as a Bluetooth® connection, a Bluetooth® low energy (BLE) connection, and/or a Wi-Fi® connection, and/or a wired connection. The networking circuitry 11, in combination with circuitry of the computational device, can be used to request, retrieve, and/or receive data from a remote server, for example. In embodiments, the circuitry can include operable connection of one or more sensors with the processor 9, or other circuitry, for performing logic operations and/or methods based on data received from the one or more sensors, for example.

As used herein, the terms “system” and “computational system” refer to one or more computational devices that are configured for performing all or part of any method of the disclosure, in any order or sequence of steps, optionally in combination with one or more other computational devices that are configured for performing all or part of any method of the disclosure, in any order or sequence of steps. In at least some instances, a method can be performed by two or more computational devices that together form at least part of a computational system, and in such instances, the steps carried out by a first computational device can be complementary to the steps carried out by a second computational device. In other instances, a method can be performed by one computational device that forms at least part of a computational system.

Devices and systems can be operably connected by one or more electronic communication connections (e.g., wired or wireless, e.g., Bluetooth®, Wifi®, infrared (IR), or the like), or network connections (e.g., intranet, internet, telecommunications network, or the like) for electronic communications that utilize one or more communication protocols for the transmission or exchange of data, for example, for carrying out all or part of a method as a computational method.

As used herein, the term “computational method” refers to a method, or a portion of a method, that is carried out substantially or entirely by a device or system, optionally with little or no human or manual input.

As used herein, the term “circuitry” refers to one or more physical, computational, electrical circuits configured to perform all or a portion of a method or process of the disclosure. Circuitry can include a processor or microprocessor configured to execute processor-executable instructions, such as software and/or firmware, for enablement of a computer program or software application that accepts inputs as data (e.g., biomarker measurement data, information about a desired beauty outcome, information about one or more possible courses of action) and computes outputs as data (e.g., responsiveness metric, recommendations, and the like). Circuitry can include, instead of or in addition to a processor or microprocessor, dedicated hardware circuitry configured to perform logic operations by way of a configuration thereof. Circuitry can be comprised as an element of a computational device or system as disclosed herein.

As used herein, the term “instructional material,” and similar terms, refers to any material or media, whether print, audio, visual, illustrative, audiovisual, tactile, or otherwise, that can convey to an individual information about how to use a kit, device, system, or other aspect of the disclosure in a method or process of the disclosure.

While various types of computational devices useful for systems of the disclosure are discussed herein or are otherwise envisioned, an example computational device can be implemented as a device on a network. Examples can include servers, personal computers, mobile phones, smart phones, tablet computers, embedded computational devices, and other devices that can be used to implement portions of embodiments of the present disclosure. Embodiments of a computational device can be implemented in or can include an application-specific integrated circuit (ASIC), a field-programmable gate array (FPGA), or other customized device. Moreover, those of ordinary skill in the art and others will recognize that the computational device can be any one of any number of currently available or yet to be developed devices.

In its most basic configuration, a computational device includes at least one processor and a system memory connected by a communication bus. Depending on the exact configuration and type of device, the system memory can be volatile or nonvolatile memory, such as read only memory (“ROM”), random access memory (“RAM”), EEPROM, flash memory, or similar memory technology. Those of ordinary skill in the art and others will recognize that system memory typically stores data and/or program modules that are immediately accessible to and/or currently being operated on by the processor. In this regard, the processor can serve as a computational center of the computational device by supporting the execution of instructions.

A computational device can include a network interface comprising one or more components for communicating with other devices over a network. Embodiments of the present disclosure can access basic services that utilize the network interface to perform communications using common network protocols. The network interface can also include a wireless network interface configured to communicate via one or more wireless communication protocols, such as Wi-Fi, 2G, 3G, LTE, WiMAX, Bluetooth, Bluetooth low energy, and/or the like. As will be appreciated by one of ordinary skill in the art, the network interface can represent one or more wireless interfaces or physical communication interfaces described and illustrated above with respect to particular components of the computational device.

The computational device also includes a storage medium. However, services can be accessed using a computational device that does not include means for persisting data to a local storage medium. Therefore, the storage medium can be optional. In any event, the storage medium can be volatile or nonvolatile, removable or nonremovable, implemented using any technology capable of storing information such as, but not limited to, a hard drive, solid state drive, CD ROM, DVD, or other disk storage, magnetic cassettes, magnetic tape, magnetic disk storage, and/or the like.

Suitable implementations of computational devices that include a processor, system memory, communication bus, storage medium, and network interface are known and commercially available. For ease of illustration and because it is not important for an understanding of the claimed subject matter, the disclosure does not show some of the typical components of many computational devices. In this regard, the computational device can include input devices, such as a keyboard, keypad, mouse, microphone, touch input device, touch screen, tablet, and/or the like. Such input devices can be coupled to the computational device by wired or wireless connections including RF, infrared, serial, parallel, Bluetooth, Bluetooth low energy, USB, or other suitable connections protocols using wireless or physical connections. Similarly, the computational device can also include output devices such as a display, speakers, printer, and the like. Since these devices are well known in the art, they are not illustrated or described further herein.

As used herein, the term “water quality marker” refers to a physical property of water that relates to its quality or sufficiency for improving or maintaining hair health, such as management of hair brittleness, breakage, frizz, dryness, dullness, stiffness, elasticity, friction, porosity, strand loss, thinning, color change, fading, brassiness, and the like. Non-limiting examples of physical properties of water that can be used as water quality markers include water hardness, magnesium concentration, calcium concentration, pH level, chlorine level, copper (I) concentration, copper (II) concentration, and the like.

As used herein, the term “cosmetic mitigation product” refers to a cosmetic product (e.g., a cosmetic composition) that, when applied to the hair or scalp of an individual, mitigates alteration or damage of the hair due to poor water quality, such as a high degree of water hardness. Cosmetic mitigation products include, but are not limited to, shampoos, conditioners, masks, and the like, and can include one or more chelating agents for removal of one or more types of metal ions, or metal-containing compounds, from the water for protection of the individual's hair.

As used herein, the term “chelating agent” refers to a metal chelating agent, e.g., a chemical compound, that binds or chelates one or more types of metal ions, or metal-containing compounds, present in a liquid such as water. Non-limiting examples of chelating agents include disodium EDTA, tetrasodium EDTA, pentasodium pentetate, sodium gluconate, sodium phytate/phytic acid, citric acid, glycine, and the like.

In example embodiments, a metal chelating agent comprises ascorbic acid, ferulic acid, phytic acid, or any combination thereof, including but not necessarily limited to as described in U.S. Patent App. Pub. No. US20220202671A1, which is incorporated by reference herein in its entirety for all purposes. In embodiments, a metal chelating agent comprises a combination of ascorbic acid, ferulic acid and phytic acid in a carrier system that includes water, and further includes one or more other ingredients for providing any of a variety of forms of products, the ingredients including but not limited to those suitable for use in a cosmetic composition such as a glycol, a humectant, a preservative, a surfactant, a thickener, an active compound, or a combination thereof. A cosmetic composition can also include emulsifiers, emollients, fragrances, pH adjusters, other cosmetically acceptable additives, or a combination thereof. For the avoidance of doubt, the cosmetic composition can include one or more of any one of the additional ingredients. For example, the cosmetic composition can include one, or more than one glycol, or one, or more than one humectant, and the like. It will be understood that additional ingredients, when present, are present in the carrier system in either a water phase or an oily phase. The cosmetic composition can be any suitable cosmetic composition, such as, but not limited to, a single phase solution comprising the water phase, a multi-phase solution comprising at least one water phase, an emulsion comprising a water phase, such as, but not limited to, a water-in-oil emulsion, an oil-in-water emulsion, or a silicone-in-water emulsion. The cosmetic composition can be provided in a product form for application to keratinous tissue, such as, but not limited to, a skin care product or a hair care product, and can be a product form such as, but not limited to, a hydroglycolic solution, a micellar water, a cream or lotion, a toner, a cleanser, or a make-up remover. The cosmetic composition in any of the product forms can be either a leave-on or a rinse-off formulation. In embodiments, the cosmetic composition according is a single phase solution that comprises one or more solutes dissolved in one or more solvents to form a substantially homogeneous liquid that appears to be substantially clear to the naked eye. In embodiments, the cosmetic composition is a single phase water solution. In embodiments, the cosmetic composition is a hydroglycolic solution that comprises one or a combination of glycols.

In example embodiments, a cosmetic composition comprises one or more N,N′-dibenzylethylenediamine-N,N′-diacetic acid derivatives that enable keratin materials to be protected against the effects of one or more metal ions, metal-containing compounds, or pollutants, including but not necessarily as described in U.S. Pat. App. Pub. No. 2004/0039059 A1, which is incorporated by reference herein in its entirety for all purposes. A N,N′-Dibenzylethylenediamine-N,N′-diacetic acid displays fewer side-effects than other complexing molecules, for instance deferoxamine, EHPG or DTPA. Furthermore, this molecule has antioxidant capacities. Consequently, a N,N′-dibenzylethylenediamine-N,N′-diacetic acid derivative molecule makes it possible to protect keratin material against the effects of pollutants and to form with iron complexes with a low association constant, which reduces the toxicological risks associated with a disruption in cell metabolism. The topical application of a N,N′-dibenzylethylenediamine-N,N′-diacetic acid derivative molecule in nonesterified form makes it possible to maintain the active agent in an extracellular environment, thus making it possible firstly to limit the risks of interaction with the intracellular divalent ions and secondly to avoid concentration, by chelating the metals originating from the pollution, in the cell. These derivatives allow better cell regeneration and also a return to homeostasis, thus making it possible to obtain healthier keratin materials.

Thus, in embodiments, the use in topical application of at least one N,N′-dibenzylethylenediamine-N,N′-diacetic acid derivative as an antipollution agent is contemplated, including as an antipollution cosmetic agent.

As used herein, the term “antipollution cosmetic agent” includes an agent that protects the hair, skin, and/or other keratin materials so as to prevent, attenuate, and/or eliminate the deleterious effects of metals.

One or more N,N′-dibenzylethylenediamine-N,N′-diacetic acid derivatives can correspond to those of formula (I):

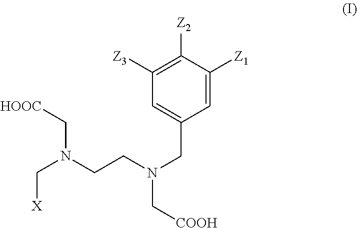

in which X represents COOH or a ring

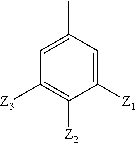

in which Z₁, Z₂, and Z₃, independently of each other, may be H, OR or R, R representing a linear or branched, saturated or unsaturated, optionally substituted C₁-C₈ alkyl radical, or a salt, a metal complex or an ester of such a derivative.

The C₁-C₈ alkyl radical is preferably a saturated C₁-C₄ radical, such as methyl, ethyl, isopropyl or tert-butyl.

Examples of possible substituents that can be mentioned include the hydroxyl group and halogens.

Salts that can be mentioned include the addition salts with a mineral or organic acid, for instance sulfuric acid, hydrochloric acid, nitric acid, phosphoric acid or acetic acid, and addition salts with a mineral or organic base, for instance sodium hydroxide, potassium hydroxide or triethanolamine.

Metal complexes that can be mentioned include the complexes formed by addition of ZnCl₂ or CaCl₂), for example.

Esters that can especially be mentioned include the methyl ester or the ethyl ester.

In embodiments, compounds that can be used as one or more N,N′-dibenzylethylenediamine-N,N′-diacetic acid derivatives can be prepared according to the methods described in PCT App. Pub. No. WO1994011338A1, which is incorporated by reference herein for all purposes.

Preferably, the derivative of formula (I) is chosen from N,N′-bis(3-hydroxybenzyl)ethylenediamine-N,N′-diacetic acid and N,N′-bis(3,4,5-trimethoxybenzyl)-ethylenediamine-N,N′-diacetic acid.

In example embodiments, derivatives of formula (I) can be used to protect keratin materials against the effects of pollutant metals or metalloid elements.

As used herein, the term “keratin materials” includes but is not necessarily limited to the skin, the scalp, the hair, the eyelashes, the eyebrows, the nails, and the mucous membranes.

In example embodiments, derivatives of formula (I) can be formulated into a cosmetic composition that contains from 0.005% to 10% and preferably 0.1% to 5% by weight of compound of formula (I), or a derivative thereof, relative to the total weight of the composition. The composition can also contain at least one other antipollution compound, including those chosen especially from anthocyans and/or derivatives thereof, compounds containing a thioether, sulfoxide or sulfone function, ergothioneine and/or its derivatives, metal-chelating agents such as antioxidants and extracts of cells of a plant of the Pontederiacea family. Among the antioxidants that can be chosen more particularly are polyphenols and, inter alia, ellagic acid.

A cosmetic composition can also comprise a cosmetically acceptable medium more particularly including water and/or optionally of a cosmetically acceptable organic solvent. The organic solvents can represent from 5% to 98% of the total weight of the composition. They can be chosen from the group consisting of hydrophilic organic solvents, lipophilic organic solvents and amphiphilic solvents, or mixtures thereof. Among the hydrophilic organic solvents that may be mentioned, for example, are mono- or polyfunctional alcohols such as linear or branched lower monoalcohols containing from 1 to 8 carbon atoms, for instance ethanol, propanol, butanol, isopropanol and isobutanol, optionally oxyethylenated polyethylene glycols containing from 6 to 80 ethylene oxides, polyols such as propylene glycol, isoprene glycol, butylene glycol, glycerol, sorbitol and its derivatives, monoalkyl or dialkyl isosorbides, the alkyl groups of which contain from 1 to 5 carbon atoms, for instance dimethyl isosorbide, glycol ethers, for instance diethylene glycol monomethyl ether or monoethyl ether, and propylene glycol ethers, for instance dipropylene glycol methyl ether. Lipophilic organic solvents that can be mentioned, for example, include fatty esters such as diisopropyl adipate, dioctyl adipate, and alkyl benzoates. Amphiphilic organic solvents that can be mentioned include polyols such as propylene glycol (PPG) derivatives, such as esters of polypropylene glycol and in particular esters of polypropylene glycol and of fatty acids, derivatives of PPG and of fatty alcohols, for instance PPG-23 oleyl ether, and PPG-36 oleate.

In embodiments, and in order for the cosmetic or dermatological composition to be more pleasant to use (softer to apply, more nourishing and more emollient), it is possible to add a fatty phase to the medium of these compositions. The fatty phase can represent from 0 to 50% relative to the total weight of the composition. This fatty phase may comprise one or more oils preferably chosen from the group consisting of: volatile or nonvolatile, linear, branched or cyclic, organomodified or non-organomodified, water-soluble or liposoluble silicones, mineral oils such as liquid paraffin and liquid petroleum jelly, oils of animal origin such as perhydrosqualene, oils of plant origin such as sweet almond oil, avocado oil, castor oil, olive oil, jojoba oil, sesame oil, groundnut oil, macadamia oil, grape-seed oil, rapeseed oil or coconut oil, synthetic oils such as purcellin oil and isoparaffins, fluoro oils and perfluoro oils, fatty acid esters such as purcellin oil.

The fatty phase can also comprise as fatty substances one or more fatty alcohols, fatty acids or waxes (paraffin wax, polyethylene wax, carnauba wax or beeswax).

Cosmetic compositions of the disclosure can also contain adjuvants that are common in the cosmetic and dermatological field, such as standard aqueous or lipophilic gelling agents and/or thickeners, hydrophilic or lipophilic active agents, preserving agents, antioxidants, fragrances, emulsifiers, moisturizers, pigmenting agents, depigmenting agents, keratolytic agents, vitamins, emollients, sequestering agents, surfactants, polymers, acidifying or basifying agents, fillers, free-radical scavengers, ceramides, sunscreens, especially ultraviolet screening agents, insect repellents, slimming agents, colorants, bactericides and antidandruff agents.

The amounts of these various adjuvants include those conventionally used in the fields under consideration.

Needless to say, a person skilled in the art will take care to select the optional compound(s) to be added to the composition according to the disclosure, such that the advantageous properties intrinsically associated with the composition in accordance with the disclosure are not, or are not substantially, adversely affected by the envisioned addition.

The compositions according to the disclosure can be in any presentation form normally used for topical application, especially in the form of an aqueous, aqueous-alcoholic or oily solution, an oil-in-water or water-in-oil or multiple emulsion, an aqueous or oily gel, a liquid, pasty or solid anhydrous product or a dispersion of oil in an aqueous phase with the aid of spherules, these spherules possibly being polymer nanoparticles such as nanospheres and nanocapsules, or better still lipid vesicles of ionic and/or nonionic type.

In embodiments, a cosmetic composition can be more or less fluid and/or can have the appearance of a white or colored cream, an ointment, a milk, a lotion, a serum, a paste or a mousse. In embodiments, a cosmetic composition can optionally be applied to the skin in aerosol form, and/or applied in solid form, and for example in the form of a stick. In embodiments, a cosmetic composition may be used as care products and/or as makeup products. In embodiments, a cosmetic composition can have a pH of between 3 and 8 and preferably between 5 and 7.

In example embodiments, a cosmetic composition comprises ellagic acid as a metal chelating agent, including but not necessarily limited to as described in U.S. Pat. App. Pub. No. 2004/0013696A1, which is incorporated by reference herein in its entirety for all purposes.

Ellagic acid, also known as 2,3,7,8-tetrahydroxy-(1)benzopyrano (5,4,3-cde) (1)benzopyran-5,10-dione, is a molecule belonging to the polyphenol group and is present in the plant kingdom. French Patent Application FR1478523A, which is incorporated by reference herein for all purposes, discloses a process for purifying ellagic acid and also the purified ellagic acids obtained by such a process.

Ellagic acid has the following chemical formula:

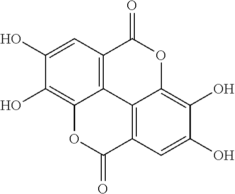 [SMILES: O=c1oc2c(O)c(O)cc3c(=O)oc4c(O)c(O)cc1c4c23]

which comprises four fused rings.

Ellagic acid, as well as its salts, its metal complexes, its monoether or polyether derivatives, its monoacyl or polyacyl derivatives and its carbonate or carbamate derivatives, derived from the hydroxyl groups, can be included in one or more cosmetic compositions of the disclosure as antipollution cosmetic agents.

In example embodiments, a cell extract of at least a plant of the family Pontederiaceae can be used in a cosmetic composition of the disclosure as a metal chelating agent or as an anti-pollution agent, including but not necessarily limited to as described in U.S. Pat. App. Pub. No. 2004/0047831A1, which is incorporated by reference herein in its entirety for all purposes.

The Pontederiaceae family includes several genera, among which is Eichhornia crassipes, also known as water hyacinth. This genus contains about seven species, including E. azurea and E. crassipes. These are aquatic or amphibious floating weeds with a procumbent rhizome and floating or submerged sheating leaves. In particular example embodiments, a cell extract of at least a plant of the species Eichhornia crassipes can be used in a cosmetic composition of the disclosure as a metal chelating agent or as an anti-pollution agent. Such a cell extract can include a root and/or leaf cell extract. Any extraction method known to those skilled in the art can be used to produce the cell extract, including but not necessarily limited to alcoholic extracts, especially ethanolic and/or methanolic extracts, and aqueous-alcoholic extracts.

As used herein, the term “lifestyle mitigation product” refers to a product, other than a cosmetic product, that can be used by the individual for mitigation of alteration or damage of the hair due to poor water quality, such as a high degree of water hardness. Examples of lifestyle mitigation products include water softener systems or devices, water treatment compositions or kits, and the like. As an example, a lifestyle mitigation product can comprise a portable water softener device that attaches to the shower or sink, such that water that passes through the portable water softener device has a decreased hardness level relative to water that does not pass through the portable water softener device. In embodiments, a lifestyle mitigation product can comprise one or more metal chelating agents therein configured for chelation of metals or metal-containing compounds, and removal of metals and/or metal-containing compounds from the water prior to use of the water.

### NON-LIMITING EMBODIMENTS

While general features of the disclosure are described and shown and particular features of the disclosure are set forth in the claims, the following non-limiting embodiments relate to features, and combinations of features, that are explicitly envisioned as being part of the disclosure. The following non-limiting Embodiments contain elements that are modular and can be combined with each other in any number, order, or combination to form a new non-limiting Embodiment, which can itself be further combined with other non-limiting Embodiments.
Embodiment 1. A system configured for management of hair health of an individual, the system comprising: circuitry configured to receive a measurement of one or more water quality markers that are relevant to the individual and compute, based on the measurement, whether a product or a service would advance the hair health of the individual toward a desired beauty outcome for a responsiveness metric; and circuitry configured to recommend or not recommend the product or the service to the individual, based on the responsiveness metric, such that the hair health of the individual is advanced toward the desired beauty outcome.
Embodiment 2. The system of Embodiment 1 or any other Embodiment, wherein the measurement of the one or more water quality markers is transmitted from a data storage computational device and received by a computational device.
Embodiment 3. The system of any one of Embodiments 1-2 or any other Embodiment, wherein the data storage computational device comprises associations of a plurality of geophysical locations and corresponding measurements of water quality markers at or near the geophysical locations, or at or near a water source thereof.
Embodiment 4. The system of any one of Embodiments 1-3 or any other Embodiment, wherein the computational device comprises a networked server, a cloud computing system, a smartphone, a tablet, a desktop computer, a laptop computer, a smart watch, a wearable device, or any combination thereof.
Embodiment 5. The system of any one of Embodiments 1-4 or any other Embodiment, wherein a first threshold of the measurement corresponds with a positive responsiveness metric and predicted responsiveness of hair of the individual to the product or the service, and wherein a second threshold of the measurement corresponds with a negative responsiveness metric and predicted non-responsiveness of hair of the individual to the product or the service.
Embodiment 6. The system of any one of Embodiments 1-5 or any other Embodiment, wherein the water quality marker comprises water hardness, and the measurement comprises a concentration of magnesium and/or a concentration of calcium in water that is relevant to the individual.
Embodiment 7. The system of any one of Embodiments 1-6 or any other Embodiment, wherein the product comprises a cosmetic mitigation product configured to mitigate hair damage due to water hardness, and wherein the cosmetic mitigation product comprises a chelating agent configured to chelate magnesium and/or calcium for removal from the water.
Embodiment 8. The system of any one of Embodiments 1-7 or any other Embodiment, wherein the cosmetic mitigation product is a shampoo, conditioner, deep conditioner, hair mask configured for moisture replenishment and/or damage repair, and/or scrub primer; and wherein the chelating agent is selected from the group including, but not necessarily limited to: disodium EDTA, tetrasodium EDTA, pentasodium pentetate, sodium gluconate, sodium phytate/phytic acid, citric acid, glycine, and any combination thereof.
Embodiment 9. The system of any one of Embodiments 1-8 or any other Embodiment, wherein the product comprises a lifestyle mitigation product configured to mitigate hair damage due to water hardness, and wherein the lifestyle mitigation product comprises a chelating agent configured to chelate magnesium and/or calcium for removal from the water.
Embodiment 10. The system of any one of Embodiments 1-9 or any other Embodiment, wherein the lifestyle mitigation product is a water softener, a shower stick, or a recommendation to reduce a frequency of hair washing and/or hair rinsing; and wherein the chelating agent is selected from the group including, but not necessarily limited to: disodium EDTA, tetrasodium EDTA, pentasodium pentetate, sodium gluconate, sodium phytate/phytic acid, citric acid, glycine, and any combination thereof.
Embodiment 11. The system of any one of Embodiments 1-10 or any other Embodiment, wherein the one or more water quality metrics are relevant to the individual due at least in part to the measurement being taken from water that is or will be used by the individual at a particular time.
Embodiment 12. The system of any one of Embodiments 1-11 or any other Embodiment, wherein the water is or will be proximal to the individual at the particular time in the course of travel or transportation.
Embodiment 13. The system of any one of Embodiments 1-12 or any other Embodiment, wherein the water quality marker comprises pH, chlorine, copper (I), copper (II), or any combination thereof, and the measurement comprises a pH level, a concentration of chlorine, a concentration of copper (I), a concentration of copper (II), or any combination thereof, in water that is relevant to the individual.
Embodiment 14. The system of any one of Embodiments 1-13 or any other Embodiment, wherein the system is configured to receive the measurement from a water testing kit that is configured for testing one or more water quality markers that are relevant to the individual.
Embodiment 15. The system of any one of Embodiments 1-14 or any other Embodiment, wherein the water testing kit comprises one or more pH colorimetric strips for measurement of pH, and/or one or more reagents for measurement of at least one water quality marker selected from the group including, but not necessarily limited to: magnesium, calcium, chlorine, copper (I), copper (II), and any combination thereof.

While illustrative embodiments have been illustrated and described, it will be appreciated that various changes can be made therein without departing from the spirit and scope of the disclosure.

1. A system configured for management of hair health of an individual, the system comprising:
circuitry configured to receive a measurement of one or more water quality markers that are relevant to the individual and compute, based on the measurement, whether a product or a service would advance the hair health of the individual toward a desired beauty outcome for a responsiveness metric; and
circuitry configured to recommend or not recommend the product or the service to the individual, based on the responsiveness metric, such that the hair health of the individual is advanced toward the desired beauty outcome.

2. The system of claim 1, wherein the measurement of the one or more water quality markers is transmitted from a data storage computational device and received by a computational device.

3. The system of claim 2, wherein the data storage computational device comprises associations of a plurality of geophysical locations and corresponding measurements of water quality markers at or near the geophysical locations, or at or near a water source thereof.

4. The system of claim 2, wherein the computational device comprises a networked server, a cloud computing system, a smartphone, a tablet, a desktop computer, a laptop computer, a smart watch, a wearable device, or any combination thereof.

5. The system of claim 1, wherein a first threshold of the measurement corresponds with a positive responsiveness metric and predicted responsiveness of hair of the individual to the product or the service, and wherein a second threshold of the measurement corresponds with a negative responsiveness metric and predicted non-responsiveness of hair of the individual to the product or the service.

6. The system of claim 1, wherein the water quality marker comprises water hardness, and the measurement comprises a concentration of magnesium and/or a concentration of calcium in water that is relevant to the individual.

7. The system of claim 6, wherein the product comprises a cosmetic mitigation product configured to mitigate hair damage due to water hardness, and wherein the cosmetic mitigation product comprises a chelating agent configured to chelate magnesium and/or calcium for removal from the water.

8. The system of claim 7, wherein the cosmetic mitigation product is a shampoo, conditioner, deep conditioner, hair mask configured for moisture replenishment and/or damage repair, and/or scrub primer; and wherein the chelating agent is selected from the group consisting of: disodium EDTA, tetrasodium EDTA, pentasodium pentetate, sodium gluconate, sodium phytate/phytic acid, citric acid, glycine, and any combination thereof.

9. The system of claim 6, wherein the product comprises a lifestyle mitigation product configured to mitigate hair damage due to water hardness, and wherein the lifestyle mitigation product comprises a chelating agent configured to chelate magnesium and/or calcium for removal from the water.

10. The system of claim 9, wherein the lifestyle mitigation product is a water softener, a shower stick, or a recommendation to reduce a frequency of hair washing and/or hair rinsing; and wherein the chelating agent is selected from the group consisting of: disodium EDTA, tetrasodium EDTA, pentasodium pentetate, sodium gluconate, sodium phytate/phytic acid, citric acid, glycine, and any combination thereof.

11. The system of claim 1, wherein the one or more water quality metrics are relevant to the individual due at least in part to the measurement being taken from water that is or will be used by the individual at a particular time.

12. The system of claim 11, wherein the water is or will be proximal to the individual at the particular time in the course of travel or transportation.

13. The system of claim 1, wherein the water quality marker comprises pH, chlorine, copper (I), copper (II), or any combination thereof, and the measurement comprises a pH level, a concentration of chlorine, a concentration of copper (I), a concentration of copper (II), or any combination thereof, in water that is relevant to the individual.

14. The system of claim 1, wherein the system is configured to receive the measurement from a water testing kit that is configured for testing one or more water quality markers that are relevant to the individual.

15. The system of claim 14, wherein the water testing kit comprises one or more pH colorimetric strips for measurement of pH, and/or one or more reagents for measurement of at least one water quality marker selected from the group consisting of: magnesium, calcium, chlorine, copper (I), copper (II), and any combination thereof.
